(19) United States
(12) Patent Application Publication
(10) Pub. No.: US 2012/0260355 A1
(43) Pub. Date: Oct. 11, 2012

(54) Method for Evaluating Inhibitory Polynucleotide Efficiency and Efficacy

(75) Inventors: Ira S. Cohen, Stony Brook, NY (US); Thomas W. White, Stony Brook, NY (US); Richard B. Robinson, Cresskill, NJ (US); Peter R. Brink, Setauket, NY (US); Richard T. Mathias, Port Jefferson, NY (US); Michael R. Rosen, New York, NY (US)

(73) Assignee: The Trustees Of Columbia University In The City Of New York, New York, NY (US)

(21) Appl. No.: 13/517,094

(22) PCT Filed: Dec. 22, 2010
(86) PCT No.: PCT/US10/61880

§ 371 Date: Jun. 19, 2012

Related U.S. Application Data
(60) Provisional application No. 61/289,325, filed on Dec. 22, 2009.

(51) Int. Cl. A61K 49/00

(52) U.S. Cl. 800/3; 424/9.2

(57) ABSTRACT

The present invention provides a method for testing the efficiency of delivering an inhibitory polynucleotide to a target cell or tissue. The invention also provides a method for testing efficiency of delivering and efficacy for an effect on tumor size of an inhibitory polynucleotide against a target gene.

### CROSS-REFERENCE TO RELATED APPLICATIONS

This application claims benefit of Provisional Application No. 61/289,325, filed Dec. 22, 2009, the entire contents of which are hereby incorporated by reference as if fully set forth herein, under 35 U.S.C. §119(e).

### STATEMENT OF GOVERNMENT INTEREST

This invention was made with government support under grant HL67101 from the National Institutes of Health. The government has certain rights in the invention.

### FIELD OF THE INVENTION

The invention relates to methods for evaluating delivery of inhibitory polynucleotides such as siRNAs and antisense RNAs for efficiency of delivery to a target cell or tissue and/or efficacy of an effect on the target cell or tissue.

### BACKGROUND OF THE INVENTION

Inhibitory polynucleotides (e.g., siRNA, shRNA, miRNA (microRNA), antisense RNA) prevent or reduce the expression of one or more target genes by directing the degradation of the target gene mRNA or by otherwise preventing translation of the target gene mRNA.

Inhibitory polynucleotides such as antisense RNA and siRNA have been useful tools in the laboratory for studying gene function and effects of inhibiting target gene expression. However, as agents in the treatment of disease, inhibitory polynucleotides have had more limited success. One barrier to the use of inhibitory polynucleotides as agents in the treatment of disease has been the difficulty in delivering inhibitory polynucleotides to target cells or tissue in order to decrease expression of a target gene. Delivery of an inhibitory polynucleotide like an siRNA to a target cell or tissue should enable the inhibitory polynucleotide to both reach the target cell or tissue and enter the cytoplasm of target cells.

### SUMMARY OF THE INVENTION

In general, in a first embodiment, the invention features a method for testing the efficiency of delivering an inhibitory polynucleotide comprising: (a) introducing target cells expressing a fluorescent protein and capable of forming a cell mass into a host animal; (b) delivering the inhibitory polynucleotide against the fluorescent protein to the host animal; and (c) measuring in vivo the fluorescent protein fluorescence over a time period to determine the efficiency of delivering the inhibitory polynucleotide to the target cell cells (or a target cell mass).

Embodiments of the invention may include one or more of the following features. In one embodiment the host animal is a nude mouse. In one embodiment, the inhibitory polynucleotide is an siRNA, shRNA, microRNA, antisense RNA, morpholino or a peptide nucleic acid. In another embodiment the inhibitory polynucleotide is delivered locally. In another embodiment the inhibitory polynucleotide is delivered systemically. In another embodiment the inhibitory polynucleotide is delivered as a naked inhibitory polynucleotide, or packaged in a liposome, nanoparticle, virus, bacteria, or in a donor cell expressing one or more connexin proteins. In one embodiment the donor cell is an immune privileged cell such as a mesenchymal stem cell (MSC). In another embodiment the inhibitory polynucleotide is complexed with cationic lipids, cholesterol, peptides, polyethyleneimine, and/or condensing polymers. In another embodiment the inhibitory polynucleotide has one or more chemical modifications including changes to the inhibitory polynucleotide backbone, replacement of one or more nucleotides with nucleotide analogues, and addition of conjugates to the polynucleotide. In another embodiment the chemical modification is inclusion of a 2′O-methyl RNA, phosphorothioate bonds, linked nucleic acids, locked nucleic acids, and/or addition of a moiety such as cholesterol, peptide, polyethylene glycol, or fatty acid. In one embodiment the fluorescent protein is GFP or a variant of GFP. In another embodiment the fluorescent protein is a far-red emitting protein such as mPlum, E2-Crimson, mRasberry and HcRed; a red protein such as mCherry, mStrawberry, AsRed2, DsRed-monomer, DsRed2, tdTomato, DsRed-Express, DsRed-Express2 and J-Red; an orange protein such as mOrange and mOrange2; a yellow protein such as mBanana and ZsYellow; a yellow-green protein such as EYFP, mCitrine, Venus and YPet; a green protein such as ZsGreen, ZsGreen1, AcGFP1; a cyan protein such as AmCyan1, Cerulean, mCFP, CyPet; or a UV excitable green protein such as T-Sapphire; and modifications of such proteins.

In one embodiment the host animal is immune-compromised and the target cell mass includes one or more pseudotumors or tumors. In an embodiment method for testing the efficiency of delivering a first inhibitory polynucleotide to an immune-compromised host animal, comprises (a) introducing target cells capable of forming a target cell mass and expressing a first fluorescent protein into the host animal at a target site; (b) delivering the first inhibitory polynucleotide against the first fluorescent protein to the host animal; and (c) measuring over a time period in vivo the first fluorescent protein fluorescence to determine the efficiency of delivering the first inhibitory polynucleotide to the target cells, wherein the efficiency is inversely proportional to a reduction in fluorescence. In an embodiment the target cells form a target cell mass before the first inhibitory polynucleotide is delivered. The embodiment can further comprise (d) delivering an expression construct comprising a second fluorescent protein to the host animal; (e) delivering a second inhibitory polynucleotide against the second fluorescent protein to the host animal; and (f) measuring over a time period in vivo the second fluorescent protein fluorescence to determine the efficiency of delivering the second inhibitory polynucleotide to the cells surrounding the target site, wherein the efficiency is inversely proportional to a reduction in second fluorescent protein fluorescence.

In another embodiment (a), (b) and (c) are performed using target cells comprise a target gene, and the method further comprises (d) delivering a second inhibitory polynucleotide against the target gene to the host animal; and (e) determining that the second inhibitory polynucleotide is successfully delivered to the target cells if the first fluorescent protein fluorescence measured in step (c) is reduced. In another embodiment this method further comprises (f) determining the effect of the second inhibitory polypeptide against target gene on the target cells relative to a control.

In other embodiments the host animal is selected from the group comprising a nude mouse, a SCID mouse, a thymectomized mouse, or an irradiated mouse. The target cells can be injected subcutaneously, intraperitoneally or at an orthotopic site. In other embodiments the inhibitory polynucleotides are delivered to the target site or at a remote site. Where two inhibitory polynucleotides are delivered, in an embodiment they are delivered at the target site or at a remote site.

In an embodiment the fluorescent protein is selected from the group comprising Green fluorescent protein; a far-red emitting protein; a red protein; an orange protein; a yellow protein; a yellow-green protein; a green protein; a cyan protein; or a UV excitable green protein; and biologically active fragments or variants thereof.

In certain embodiments the target gene is expressed endogenously in the target cells. In some embodiments the target cells have been engineered to express the target gene. In some embodiments the target gene is expressed from a plasmid, or from vectors including retrovirus, lentivirus, adenovirus, and adeno-associated virus based systems. In some embodiments the target cells are tumor-forming cells and the target cell mass comprises one or more tumors.

In an embodiment the target cells expresses a second fluorescent protein and a target gene. In an embodiment, the target cells form a target cell mass, and the change in the size, growth, or metastasis of the target cell mass is determined by in vivo imaging of the second fluorescent protein.

In an embodiment the target cells express the first fluorescent protein and a target gene on the same messenger RNA, and the method includes (a)-(c) and further comprises (d) determining that a reduction in first fluorescent protein fluorescence indicates a reduction in expression of the target gene. In an embodiment the first fluorescent protein and the target gene are expressed as a fusion protein in the target cells, and are optionally separated by an IRES element on the messenger RNA. The target cells optionally also express a second fluorescent protein.

In another embodiment the host animal is an immune-compromised transgenic animal that expresses a second fluorescent protein, and the method comprises (a)-(c) and further comprises (d) delivering a second inhibitory polynucleotide against the second fluorescent protein to the host; and (e) measuring over a time period in vivo the second fluorescent protein fluorescence to determine the efficiency of delivering the second inhibitory polynucleotide to the host animal cells, wherein the efficiency is inversely proportional to a reduction in second fluorescent protein fluorescence.

In another aspect the invention provides a method for screening inhibitory polynucleotides against a target gene for an effect on a target cell mass comprising: (a) introducing target cells expressing a fluorescent protein into a host animal; (b) delivering an inhibitory polynucleotide against a target gene to the host animal; and (c) measuring in vivo the fluorescent protein fluorescence over a time period to determine effect on target cell mass.

### BRIEF DESCRIPTION OF THE DRAWINGS

The present invention is illustrated by way of example, and not by way of limitation, in the figures of the accompanying drawings and in which like reference numerals refer to similar elements.

FIG. 1: HEK293 cells stably expressing GFP were cultured to confluence. 10 million cells were concentrated into a 500 uL volume and injected by syringe with a 28 gauge needle subcutaneously into a nude mouse. Using a Maestro imaging system the live animal and fluorescent cell mass were monitored over time. FIG. 1A shows the nude mouse at T=3 hours in normal light; FIG. 1B shows the same mouse at T=3 hours using a light wave length that causes GFP to fluoresce. FIG. 1C shows another image taken 120 hour later of the same animal FIG. 1D illustrates the fluorescence intensity of GFP within the injected cell mass at various time points. FIG. 1E is the measured area of fluorescence relative to the first time point within minutes of injection.

### DETAILED DESCRIPTION OF THE INVENTION

The present inventions include various embodiments of methods for testing the efficiency of any method for delivering an inhibitory polynucleotide (e.g., shRNA, shRNA, miRNA, antisense RNA, or morpholino (non-degradable polynucleotide) to a target cell, including a normal or a diseased cell or tissue, in an immune-compromised host animal such as a nude mouse. In an embodiment, a method for determining the efficiency of delivering an inhibitory polynucleotide includes: (a) introducing target cells capable of forming a target cell mass and expressing a first fluorescent protein into the host animal at a target site; (b) delivering the first inhibitory polynucleotide against the first fluorescent protein to the host animal (either locally at the target site or at a remote site) and (c) measuring over a time period in vivo the first fluorescent protein fluorescence to determine the efficiency of delivering the first inhibitory polynucleotide to the target cells, wherein the efficiency is inversely proportional to a reduction in fluorescence. By remote site is meant injecting the inhibitory polynucleotide intravenously into the host animal, which is a form of systemic administration.

The inhibitory polynucleotides can be delivered simultaneously with the target cells, but they are typically delivered after the target cell mass has begun to form or has formed. The immune-compromised host enables the assays to be performed using heterologous target cells from a different species. Syngenic animal models may also be used such that the host animal need not be immune-compromised (Talmadge et al. (2007) Murine Models to Evaluate Novel and Conventional Therapeutic Strategies for Cancer. Am. J. of Path. 170(3): 793-804).

Any method for delivering the inhibitory polynucleotides to the target cells can be tested using the present methods. Other embodiments described below also permit a determination of the efficiency of delivering an inhibitory polynucleotide against a target gene or messenger RNA transcribed from the target gene that is expressed by the target cells.

By “inhibitory polynucleotide against” a fluorescent protein or against a target gene, is meant an inhibitory polynucleotide that inhibits or reduces the expression of the fluorescent protein or target gene. Examples of inhibitory polynucleotides for the purpose of this invention include any polynucleotide that reduces the expression of the fluorescent protein or target gene, including siRNA, shRNA, miRNA, antisense RNA, morpholino or a peptide nucleic acids (terms that are well known in the art).

In another embodiment the efficiencies of multiple methods of delivering inhibitory polynucleotides to the target cells are compared to identify the most efficient method of delivery. This method includes (a) introducing target cells capable of forming a target cell mass and expressing a first fluorescent protein into immune-compromised host animals at a target site; (b) delivering the first inhibitory polynucleotide against the first fluorescent protein to each host animal; (c) measuring over a time period in vivo the first fluorescent protein fluorescence in each host animal to determine the efficiency of delivering the first inhibitory polynucleotide to the target cells, wherein the efficiency is inversely proportional to a reduction in fluorescence and (d) selecting the most efficient method for of delivering the first inhibitory polynucleotide to the target cells.

The preferred mode of introducing the target cells is by injection, which may be subcutaneous, intraperitoneal, or at an orthotopic site. Typically, about 1 million to about 25 million target cells are injected to form a cell mass of between about 1.5 mm to about 1.5 cm in diameter. The number of target cells injected is highly variable depending upon the cell volume and the cell type of the target cells, the type of therapy, the targeted protein to be inhibited by the inhibitory polynucleotide in the target cells, and its turnover time in the cell. Routine experimentation will determine the optimum number. If tumor cells are used, a cell mass typically develops in about 2-8 weeks, depending on the type of tumor cells and the number introduced. The target cell mass may be a pseudotumor or tumor.

The size of the target cell mass can be determined histologically or preferably by imaging the fluorescence emitted by the fluorescent protein in the target cell mass using an in vivo imaging machine. In vivo imaging of fluorescent signals can be accomplished using commercially available apparatus, for example, the Maestro EX or Maestro 2 from CRi (Woburn, Mass.). Any immune-compromised host animal (i.e. immune-suppressed or immune-deficient) capable of supporting the target cell mass can be used in the present methods; preferred host animals also permit in vivo detection of fluorescence from the target cell mass, including for example, a genetically athymic “nude” mouse (see, e.g., Giovanella et al., (1974) J. Natl. Cancer Inst. 52:921), a SCID mouse, a thymectomized mouse, or an irradiated mouse (see, e.g., Bradley et al., (1978). Br. J. Cancer 38:263; Selby et al., (1980). Br. J. Cancer 41:52) can be used as a host. Xenograft tumor models are widely used to study human diseases in non-human mammals and are well known in the art.

In vivo imaging of host animals allows for the high-throughput analysis of efficiency of delivery of inhibitory polynucleotide and for determining the effects of a target gene on one or more properties of the target cells or the target cell mass (i.e., size, metastasis) as is described in more detail below.

Delivery of inhibitory polynucleotides in the various embodiments of the methods may be local (i.e., to the site of the cell mass or affected tissue) or to a remote site, (i.e. systemic, delivery to the circulatory or lymphatic systems). Local injection avoids many of the difficulties associated with intravenous administration, such as rapid elimination. The inhibitory polynucleotides can be delivered as a naked inhibitory polynucleotide, or they can be packaged in a liposome, nanoparticle, virus, bacteria, or in a donor cell such as an MSC that expresses one or more connexin proteins. Donor cells represent a cell-based delivery method wherein the inhibitory polynucleotide are present in or expressed by the donor cells. MSCs are also immune-privileged cells, which is an advantage. MSCs home to the target cells and form gap junctions with them through which the inhibitory polynucleotides are delivered. For example, MSCs expressing shRNA that specifically inhibits expression of GFP from a lentiviral vector integrated into the genome of the target cells can be injected into the tail vein of the nude mouse to test homing ability (Khahoo et al. 2006: Potapova et al., 2008) of the stem cells or any other potentially useful donor cell to home to a site and reduce GFP fluorescence. (see, e.g., Xie, F. Y., et al. (2006). Harnessing in vivo siRNA delivery for drug discovery and therapeutic development. Drug Discovery Today, 11:67-73; Oliveira, S. et al. (2006) Targeted Delivery of siRNA. J. Biomed. Biotech. 2006:1-9; Whitehead, K. A., et al. (2009) Knocking Down Barriers; Advances in siRNA Delivery. Nature Reviews, 8:129-138).

The inhibitory polynucleotides may be targeted to the target cells and the target cell mass by associating or binding the inhibitory polynucleotides with a targeting molecule such as an antibody or a cell-penetrating peptide, a ligand for a target cell surface receptor, a target cell surface antigen including a tumor antigen. The targeting molecule may be linked to the inhibitory polynucleotides by a covalent bond or may be associated ionically or by integration into the targeting mechanism (e.g., as part of the liposome, nanoparticle, or expressed on the surface of a donor cell).

In some embodiments the inhibitory polynucleotides are complexed with cationic lipids, cholesterol, peptides, polyethyleneimine, and/or condensing polymers including biopolymers. The inhibitory polynucleotides can also optionally have one or more chemical modifications selected from the group comprising changes to the inhibitory polynucleotide backbone, replacement of one or more nucleotides with nucleotide analogues, and addition of conjugates to the polynucleotides. Other modifications to the inhibitory polynucleotides are described in more detail below. Helper molecules (for example, cationic lipids or polymers, including biopolymers) or physical methods (for example electroporation, sonoporation, or hydrodynamic pressure) facilitate intracellular entrance of the inhibitory into the target cells. In other embodiments local production of inhibitory polynucleotides (such as siRNA) is increased by introducing an expression vector carrying a gene encoding the inhibitory polynucleotide (shRNA) in order to ensure prolonged levels of expression in the target cells.

By expression vector, otherwise known as an expression construct, is meant a plasmid that is used to introduce a specific gene into a target cell. Once the expression vector is inside the cell, the protein (or inhibitory RNA) that is encoded by the gene is produced by the cellular-transcription and translation machinery ribosomal complexes. The plasmid can be and often is engineered to contain regulatory sequences that act as enhancer and promoter regions and lead to efficient transcription of the gene carried on the expression vector, and large amounts of stable messenger RNA, and therefore proteins. Any expression construct can be used in the present invention, for example, viral vectors including adenoviral, lentivirus, adeno-associated viruses, or retroviral vectors. Nonviral substances such as Ormosil have also been used as expression vectors and can deliver DNA loads to specifically targeted cells in living animals. (Ormosil stands for organically modified silica or silicate.)

Any fluorescent protein or a biologically active fragment or variant thereof can be used in the embodiments of the invention. Examples of fluorescent proteins include Green fluorescent protein; a far-red emitting protein such as mPlum, E2-Crimson, mRasberry and HcRed; a red protein such as mCherry, mStrawberry, AsRed2, DsRed-monomer, DsRed2, tdTomato, DsRed-Express, DsRed-Express2 and J-Red; an orange protein such as mOrange and mOrange2; a yellow protein such as mBanana and ZsYellow; a yellow-green protein such as EYFP, mCitrine, Venus and YPet; a green protein such as ZsGreen, ZsGreen1, AcGFP1; a cyan protein such as AmCyan1, Cerulean, mCFP, CyPet; or a UV excitable green protein such as T-Sapphire; and modifications of such proteins.

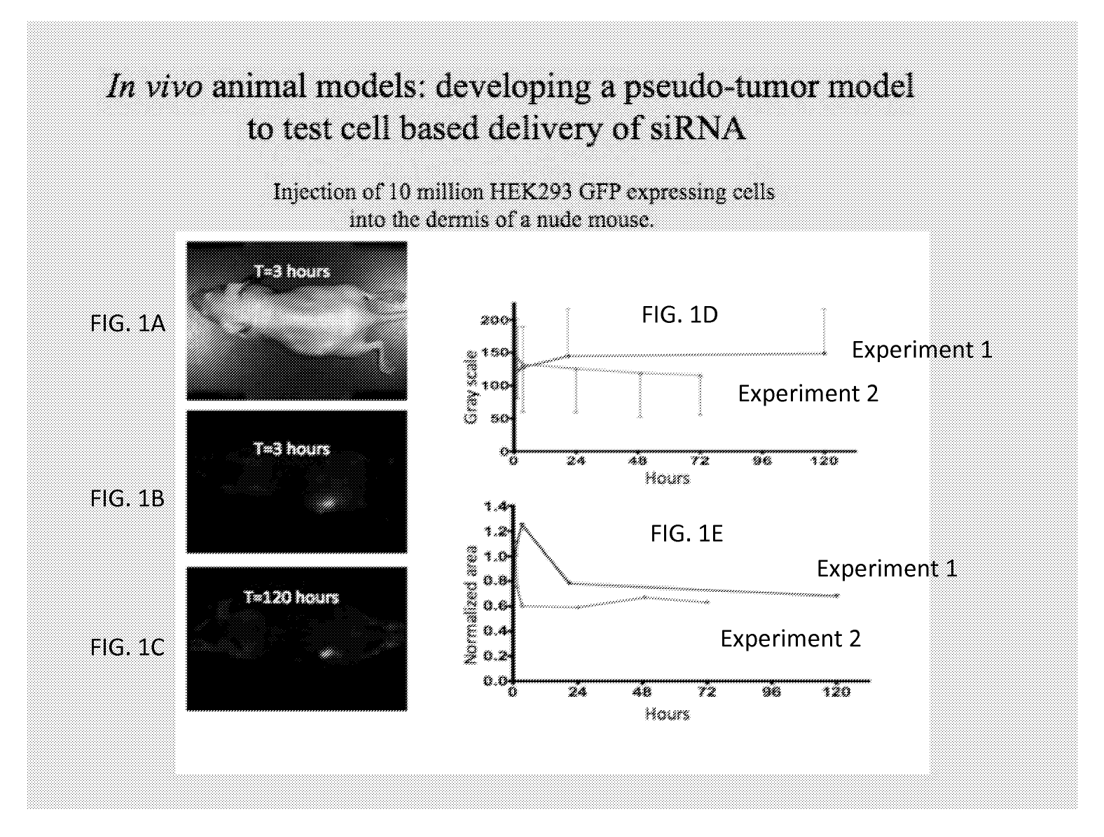

FIG. 1 shows the results of experiments that establish the validity of the model system for testing the efficiency of delivery of inhibitory polynucleotide to target cells. HEK293 target cells stably expressing GFP were cultured to confluence. About 10 million cells were concentrated into a 500 uL volume and injected by syringe with a 28 gauge needle subcutaneously into a target site of a nude mouse. Using a Maestro in vivo fluorescent imaging system the live animal and fluorescent cell mass were monitored over time. FIG. 1A shows the nude mouse at T=3 hours in normal light; FIG. 1B shows the same mouse at T=3 hours using a light wave length that causes GFP to fluoresce at the injection site of the cell mass. FIG. 1C shows another image taken 120 hour later of the same animal The results show that in this model, GFP is expressed in the target cells that formed a stable cell mass at the injection site at detectable levels by T=3 hours post injection. There was some oscillation in fluorescence over the first 24 hours, but the levels reached equilibrium after that and remained stable through the end of the experiments at T=120 hours. FIG. 1D illustrates the fluorescence intensity of GFP within the injected target cells and target cell mass at various time points in two different experiments. The top line represents experiment 1, and the bottom line represents experiment 2. FIG. 1E is the measured area of fluorescence relative to the first time point within minutes of injection and extending 72-120 hours post injection. The kinetics of fluorescent protein expression will vary depending on the type of target cell being injected, as synthesis and degradation will be cell type specific. FIG. 1E shows fluorescence intensity takes about 24 hrs to reach steady state for HEK293 cells and GFP.

In another embodiment, the invention provides a method for testing the efficiency of delivering an inhibitory polynucleotide to a target cell or tissue while monitoring the extent to which the inhibitory polynucleotide is also delivered to tissue surrounding the target cells or the target cell mass. The method for testing the efficiency of delivering an inhibitory polynucleotide includes, (a) introducing target cells capable of forming a target cell mass and expressing a first fluorescent protein into the host animal at a target site; (b) injecting an expression construct comprising a second fluorescent protein to the host animal at the target site; (c) delivering inhibitory polynucleotides against the first and second fluorescent protein to the host animal either simultaneously or within 30 minutes of one another; (d) measuring over a time period the in vivo fluorescence of the first fluorescent protein to determine the efficiency of delivering the first inhibitory polynucleotides to the target cells, wherein the efficiency is inversely proportional to a reduction in fluorescence; and (e) measuring over a time period the in vivo fluorescence of the second fluorescent protein to determine delivery of the second inhibitory polynucleotides to the surrounding tissue, wherein the efficiency is inversely proportional to a reduction in fluorescence. Delivering both siRNA's will show that if the uptake is specific only the target cell fluorescence will decline. If it is non-specific then surround cell fluorescence will also decline.

In one embodiment both of the inhibitory polynucleotides that inhibit the first and second fluorescent proteins are of the same type, for example, both are siRNAs; however this is not a requirement. At various times following introduction (delivery) of the inhibitory polynucleotides, the animal is monitored using an in vivo fluorescent imager in order to detect the fluorescence of the first fluorescent protein from the target cells and fluorescence of the second fluorescent protein from the surrounding tissue. The first and second fluorescence proteins must fluoresce at different wavelengths to be successfully imaged for high throughput analysis using the in vivo imaging machine. The amount of reduction of fluorescence of the second fluorescent protein from the tissue surrounding the cell mass indicates the efficiency of delivering the inhibitory polynucleotides to the surrounding tissue.

In another embodiment, the invention provides a method for testing the efficiency of delivering an inhibitory polynucleotide against a known target gene that is expressed by the target cells. The target gene is one for which the gene, cDNA and mRNA sequences are publically available, for example through GeneBank, or otherwise known to the user. Methods for designing and making inhibitory polynucleotides against the selected target gene are well known in the art. As before, target cells expressing a first fluorescent protein that are capable of forming a target cell mass are injected into the host animal; in some embodiments the target cells are allowed to form a target cell mass before the polynucleotides are delivered, which mass may be a tumor. An inhibitory polynucleotide against the first fluorescent protein and an inhibitory polynucleotide against the known target gene are introduced into the host animal at the target site or at a remote site either simultaneously or separately within 30 minutes of each other. The fluorescence of the targeted cells is monitored in vivo in the live host animal over a period of time by fluorescent imaging. Reduction in the fluorescence of the target cells demonstrates the efficiency of delivering the polynucleotide against the first fluorescent protein to the target cells. The inhibitory polynucleotide against the known target gene is assumed to be delivered to the target cells if the inhibitory polynucleotide against the fluorescent protein is delivered (i.e. reduces first fluorescent protein fluorescence). In some embodiments the inhibitory polynucleotides are of the same type, i.e. both siRNA or both antisense, however this is not a requirement.

In an embodiment the method for testing efficiency of delivering and efficacy for an effect on a target cell mass of an inhibitory polynucleotide against a target gene includes, includes (a) introducing target cells comprising the target gene of interest which cells are capable of forming a target cell mass and express a first fluorescent protein into the host animal at a target site; (b) delivering the first inhibitory polynucleotide against the first fluorescent protein to the host animal; (c) over a time period in vivo measuring the first fluorescent protein fluorescence to determine the efficiency of delivering the first inhibitory polynucleotide to the target cells, wherein the efficiency is inversely proportional to a reduction in fluorescence; (d) delivering a second inhibitory polynucleotide against the target gene to the host animal; and (e) determining that the second inhibitory polynucleotide is successfully delivered to the target cells if the first fluorescent protein fluorescence measured in step (c) is reduced. This method can further comprise (f) determining the effect of the second inhibitory polypeptide against target gene on the target cells relative to a control. For example, the effect could be a reduction in size of the target cell mass, prevention or reduction in the growth of the target cell mass, or prevention or reduction in metastasis from the target cell mass. This method can be used to screen inhibitory polynucleotides against a target gene for an effect on a target cell property.

A similar embodiment is a method for testing efficiency of delivering and efficacy for an effect on a target cell mass of an inhibitory polynucleotide against a target gene comprising: (a) introducing target cells expressing a fluorescent protein and a target gene which cells are capable of forming a cell mass into a host animal; (b) delivering an inhibitory polynucleotide against the fluorescent protein and an inhibitory polynucleotide against the target gene to the host animal; (c) measuring in vivo the fluorescent protein fluorescence over a time period to determine efficiency of delivering to the target cell mass the inhibitory polynucleotide against the target gene, wherein a reduction of fluorescence is inversely proportional to efficiency of delivering the inhibitory polynucleotide against the target gene; and (d) determining the effect on the target cell mass relative to a control.

In another embodiment, the delivery of an inhibitory polynucleotide is measured in vivo by reduction in fluorescent protein signal due to delivery of the inhibitory polynucleotide against the target gene wherein the target gene and the fluorescent protein are expressed on a single message (i.e. a single messenger RNA) as a fusion protein in the target cells. Thus, the inhibitory polynucleotide against the target gene would direct degradation (or suppression or reduction of translation) of the target gene message, which message also encodes the fluorescent protein. In another embodiment, the target gene and the fluorescent protein are expressed on a single message separated by an IRES element.

An embodiment of the invention is directed to a method for determining that delivery of an inhibitory polynucleotide against a first fluorescent protein indicates a reduction in expression of a target gene, including (a) introducing target cells capable of forming a target cell mass and expressing a first fluorescent protein and a target gene on the same messenger RNA into the host animal at a target site; (b) delivering the first inhibitory polynucleotide against the first fluorescent protein to the host animal; (c) measuring over a time period in vivo the first fluorescent protein fluorescence to determine the efficiency of delivering the first inhibitory polynucleotide to the target cells, wherein the efficiency is inversely proportional to a reduction in fluorescence, and (d) determining that a reduction in first fluorescent protein fluorescence indicates a reduction in expression of the target gene.

The reduction of the fluorescent protein signal from the target cells as described in any of the methods of the present invention identifies host animals with successful delivery of one or more inhibitory polynucleotides to the target cells. Such host animals can then utilized to determine the efficacy of the inhibitory polynucleotide against a target gene on an effect such as reduction in size of the target cell mass, prevention or reduction in the growth of the target cell mass, or prevention or reduction in metastasis from the target cell mass. In one embodiment, the size of the cell mass is determined by histology.

In another embodiment a method for screening inhibitory polynucleotides against a target gene for an effect on the target cells that includes introducing target cells expressing a fluorescent protein and a target gene on the same message into a host animal; and delivering an inhibitory polynucleotide against the target gene to the host animal. If the inhibitory polynucleotide is efficiently delivered then the fluorescence of the fluorescent protein will be reduced even though the polynucleotide is against the target gene because the target gene and the fluorescent protein are on the same messenger RNA. The in vivo the fluorescent protein fluorescence is measured over a time period to determine the efficiency of delivering the inhibitory polynucleotide, and (d) if there is a reduction in fluorescent protein fluorescence, determining that effect of the target gene expression was reduced and assessing the effect of inhibiting the target gene on the target cells and target cell mass.

In another embodiment the target cells express two different fluorescent proteins that emit fluorescence at two different wavelengths (two color technique). One fluorescent protein is used to monitor efficiency of delivery of the inhibitory polynucleotide (for example the protein is GFP and one inhibitory polynucleotide is against GFP). The other fluorescent protein could, for example, be co expressed with a target gene or used to monitor effects of the inhibitory polynucleotide against a target gene on the target cells. For example, fluorescent signal from the second fluorescent protein may be used to monitor the size of the cell mass, and/or metastasis by scanning the animal for fluorescence emitted from the second fluorescent protein.

In another example embodiment, the host cells express a target gene that will cause the cells to die if it is suppressed, and two florescent proteins, red and green. An inhibitory polynucleotide such as siRNA against the green protein is delivered together with siRNA against the non-fluorescent target gene. If there is a reduction in green fluorescence, it is assumed that both siRNA's are taken up equally effectively. If the siRNA against the target gene is effectively delivered, then the green fluorescence should decline if the siRNA against the target gene inhibits translation of the target protein. Since the target protein is an essential protein, the cell will die if it is not expressed and there should be a commensurate decline of the red fluorescence. However, if the siRNA against the target gene is not able to reduce expression of the target protein for some reason, then the green fluorescence will decline but the red will not decline. This is a means of testing the effectiveness of an siRNA that should kill a target cell once it is effectively delivered to the target cell, which is determined by the reduction in green fluorescence.

In another embodiment the invention provides a method for testing the efficiency of delivering a first inhibitory polynucleotide to a target cell while monitoring the extent to which a second inhibitory polynucleotide is delivered to other tissues of an immune-compromised transgenic host animal. In this embodiment a transgenic host animal, such as a nude mouse, that expresses the second fluorescent protein throughout the animal is utilized (see e.g., Yang et al. (2004) Transgenic Nude Mouse with Ubiquitous Green Fluorescent Protein Expression as a Host for Human Tumors. Cancer Research, 64(23): 8651-8656). By “transgenic” is meant any animal which includes a nucleic acid sequence which is inserted by artifice into a cell and becomes a part of the genome of the animal that develops from that cell. Such a transgene may be partly or entirely heterologous to the transgenic animal. Although transgenic mice represent a preferred embodiment of the invention, other transgenic mammals including, without limitation, transgenic rodents (for example, hamsters, guinea pigs, rabbits, and rats), and transgenic pigs, cattle, sheep, and goats are included in the definition. Transgenic animals carry a segment of foreign DNA that has been incorporated into their genome via non-homologous recombination (e.g., pronuclear microinjection), insertion via infection with a retroviral vector, or in some cases, by homologous insertion.

In this embodiment target cells expressing a first fluorescent protein are injected into the host animal The target cells can be allowed to begin forming a cell mass or a cell mass, tumor or pseudotumor can have been formed at the injection site before delivery of the inhibitory polypeptides, but this is not required. Inhibitory polynucleotides against the first fluorescent protein expressed by the target cells and against the second fluorescent protein expressed throughout the cells in the transgenic animal are delivered at the target site or at a remote site either simultaneously or within 30 minutes of one another by any method to the transgenic host. Following delivery of the inhibitory polynucleotides, the host animal is monitored using an in vivo fluorescent imager. The amount of reduction in fluorescence of the first fluorescent protein from the target cells or the target cell mass indicates the efficiency of delivery of the inhibitory polynucleotides to the targeted cells or the target cell mass, and the amount of reduction of fluorescence of the second fluorescent protein to other sites throughout the body indicates the efficiency of delivery of the inhibitory polynucleotides to those other sites.

Inhibitory polynucleotides are polynucleotides or polynucleotide analogs that are complimentary to a portion of a target gene or to messenger RNA encoding a protein of interest (or a biologically active fragment or variant thereof) and reduce or inhibit expression of the target gene product (e.g., mRNA or protein). Inhibitory polynucleotides are typically greater than 10 bases or base pairs in length and are composed of ribonucleotides or deoxynucleotides or a modified form of either type of nucleotide, and may be single and/or double stranded Inhibitory polynucleotides may be modified to increase stability and/or enhance delivery of the inhibitory polynucleotides to the cytosol of target cells. Inhibitory polynucleotides include chemically modified polynucleotides. Chemical modification includes changes to the inhibitory polynucleotide backbone, replacement of one or more nucleotides with nucleotide analogues, and addition of conjugates to the polynucleotide. Thus, modifications include 2′O-methyl RNA, phosphorothioate bonds, locked nucleic acids as well as addition of moieties such as cholesterol, peptides, polyethylene glycol, and fatty acids.

Polynucleotide analogs include, but are not limited to peptide nucleic acids (PNAs) and morpholinos. PNAs comprise naturally-occurring DNA or RNA bases (i.e., adenine, thymine, cytosine, guanine, and uracil) or artificial bases (i.e., bromothymine, azaadenines, azaguanines) attached to a peptide backbone through a suitable linker (e.g., amide, thioamide, sulfinamide or sulfonamide linkages). PNAs bind complementary DNA or RNA strands and can be utilized in a manner similar to antisense oligonucleotides to block the translation of specific target mRNA transcripts. PNA oligomers can be prepared according to the method provided by U.S. Pat. No. 6,713,602. U.S. Pat. No. 6,723,560 describes methods for modulating transcription and translation using sense and antisense PNA oligomers, respectively. PNAs may be obtained from commercial sources such as Panagene, Inc. PNAs are typically about 10 to about 30 subunits in length. The PNAs may also be about 15 to about 25 subunits in length. The PNAs may also be about 14 to about 20 subunits in length. The PNAs may also be about 16 to about 18 subunits in length.

Morpholino oligomers are short chains of about 10 to about 30 morpholino subunits. Morpholinos may also be about 15 to about 25, or about 18 to about 22 subunits long. Each subunit is comprised of a nucleic acid base, a morpholine ring and a non-ionic phosphorodiamidate intersubunit linkage. Morpholinos do not degrade their RNA targets, but instead act via a steric blocking mechanism. Systemic delivery into cells in adult organisms can be accomplished by using covalent conjugates of Morpholino oligonucleotides with cell penetrating peptides. An octa-guanidinium dendrimer attached to the end of a Morpholino can deliver the modified oligonucleotide (called a Vivo-Morpholino) from the blood to the cytosol. (Moulton, J. D., Jiang S. (2009). Gene Knockdowns in Adult Animals: PPMOs and Vivo-Morpholinos. Molecules, 14 (3): 1304-23; Morcos, P. A., Li Y. F., Jiang S. (2008). Vivo-Morpholinos: A non-peptide transporter delivers Morpholinos into a wide array of mouse tissues. BioTechniques 45 (6):616-26).

An inhibitory polynucleotide is complimentary or partially complimentary to the target gene or the corresponding mRNA encoding the protein of interest, or a biologically active fragment or variant thereof. Antisense RNA or antisense DNA can bind to either a gene (reducing transcription) or mRNA (reducing translation) encoding a protein of interest. Where the inhibitory polynucleotide is complimentary or partially complimentary region of the mRNA encoded by target gene, the region may be in the in the 5′ untranslated region (UTR), 3′ UTR, and/or in the coding region. An inhibitory polynucleotide may induce RNA interference (RNAi), which is a mechanism of gene-specific silencing that employs sequence-specific small interfering RNA (siRNA) to target and degrade the target gene-specific mRNA prior to translation. siRNAs are double-stranded RNA molecules, typically about 19 to about 23 nucleotides in length and having a 2 nucleotide overhang at the 3′ end of each strand. Methods for designing specific siRNAs based on an mRNA sequence are well known in the art (see e.g., Brummelkamp, T. R. et al. (2002) A system for stable expression of short interfering RNAs in mammalian cells. Science 19, 550-553; Ui-Tei, K. et al. (2004) Guidelines for the selection of highly effective siRNA sequences for mammalian and chick RNA interference. Nucleic Acids Res. 32, 936-948; Hohjoh H. (2004) Enhancement of RNAi activity by improved siRNA duplexes. FEBS Lett. 557, 193-8; and Yuan, B., et al. siRNA Selection Server: an automated siRNA oligonucleotide prediction server. (2004) Nucleic Acids Res. 32, W130-134). In addition, design algorithms are available on the websites of many commercial vendors that synthesize siRNAs, including Ambion, Clontech, Dharmacon, GenScript, and Qiagen.

Small interfering RNAs can be expressed in the form of short, hairpin loop polynucleotides known as short hairpin RNAs (shRNAs) comprising the siRNA sequence of interest and a hairpin loop segment that silence gene expression via RNA interference. Short hairpin RNAs are available through commercial vendors, which often provide online algorithms useful for designing shRNAs (e.g., Clontech, Invitrogen, ExpressOn, Gene Link, and BD Biosciences). When expressed in a cell, shRNA is rapidly processed by intracellular machinery into siRNA. Expression of shRNAs may be accomplished by ligating the DNA sequence corresponding to the shRNA into an expression construct, for example the cloning site of a double-stranded RNA (dsRNA) expression vector. Expression may be driven by RNA polymerase III promoters such as a U6 or H1 promoter. This vector is usually passed on to daughter cells, allowing the gene silencing to be inherited. Expression vectors may be plasmid vectors including retrovirus, lentivirus, adenovirus, and adeno-associated virus based systems. Vectors for expression of shRNAs are well known in the art and many are commercially available from vendors such as Clontech, Invitrogen, Millipore, Gene Therapy Systems, Ambion and Stratagene. Methods for DNA and RNA manipulations, including ligation and purification, are well known to those skilled in the art (See e.g., Sambrook, J. and Russel, D. W., (2001) Molecular Cloning: A Laboratory Manual, Third Edition. Cold Spring Harbor Laboratory Press, Cold Spring Harbor, N.Y.; and Current Protocols in Molecular Biology, (2001) John Wiley & Sons, Inc.).

In one embodiment of present invention, a donor cell expressing one or more connexin proteins (such as MSCs) is used to deliver one or more inhibitory polynucleotides to target cells. Donor cells can naturally produce one or more endogenous connexins that enable gap junction formation, or cells can be engineered to express one or more connexins, including fibroblasts, myoblasts, cardiomyocytes, skeletal muscle cells, and endothelial cells or other cells. In one embodiment the donor cell is an immune privileged cell. In one embodiment the donor cell is an immune privileged MSC that is capable of forming gap junctions comprising connexin 43 (Cx 43) with target cells through which inhibitory polynucleotides such as siRNA and morpholinos can pass (Valiunas, V., et al. (2005) Connexin-Specific Cell-to-Cell Transfer of Short Interfering RNA by Gap Junctions. J. Physiol. 568:459-468). Thus, use of a donor cells capable of forming gap junctions with target cells provides a method for the delivery of inhibitory polynucleotides to the cytosol of target cells without exposing the inhibitory polynucleotides to the extracellular space. The donor cells may be engineered to express one or more inhibitory polynucleotides as described herein. Alternatively, the donor cell may be loaded with one or more inhibitory polynucleotides for example by electroporation, prior to introducing them to an animal.

A target gene expressed in the target cells may be any gene that is up-regulated in a diseased cell, for example cancer (e.g., an oncogene) or any other gene of interest where the reduction of the target gene expression is desired. Reducing expression of some gene products may facilitate a phenotype change, for example. Such target cells may be a cancer cell line, cells derived from tumors, and/or cells expressing one or more genes that causes the target cells to be tumorigenic. The target gene may be expressed endogenously by the target cell, may be induced by exposing the target cell to an inducing agent, or may be expressed in the target cell from a construct such as a plasmid expression vector or viral construct including retrovirus, lentivirus, adenovirus, and adeno-associated virus-based systems. Expression plasmids and viral-based systems are commercially available from a large number of vendors including Clontech, Stratagene, and Invitrogen.

The target cells of the present invention are labeled so that their presence can be monitored in vivo. Cells may be labeled by expression of one or more fluorescent proteins as is described above, or by incorporating quantum dots, nanoparticles, vital dyes or a non-lethal fluorescent marker (that stay in the cytoplasm without interfering with cell function, for example Lucifer yellow), or other markers in order to monitor the cells and their progeny. Fluorescent proteins include biologically active fragments and variants thereof, such as green fluorescent protein (GFP), and modifications thereof, such as enhanced GFP and Emerald (all the green fluorescent proteins are referred to herein as GFP). Other fluorescent proteins include far-red proteins such as mPlum, E2-Crimson, mRasberry and HcRed; red proteins such as mCherry, mStrawberry, AsRed2, DsRed-monomer, DsRed2, tdTomato, DsRed-Express, DsRed-Express2 and J-Red; orange proteins such as mOrange and mOrange2; yellow proteins such as mBanana and ZsYellow, yellow-green proteins such as EYFP, mCitrine, Venus and YPet; green proteins such as ZsGreen, ZsGreen1, AcGFP1; cyan proteins such as AmCyan1, Cerulean, mCFP, CyPet; and UV excitable green proteins such as T-Sapphire; and modifications of such proteins. Vectors expressing these fluorescent proteins are available commercially from companies including Invitrogen, and Clontech. One of skill in the art can select two fluorescent proteins that are distinguishable from each other and any natural fluorescence of the surrounding tissue.

As some of the fluorescent proteins described above are structurally related, it is possible to target two related fluorescent proteins with a single inhibitory polynucleotide. Alternatively, two fluorescent proteins and the inhibitory polynucleotides useful in targeting the two fluorescent proteins may be selected to prevent any inhibition of a fluorescent protein by an inhibitory polynucleotide against a different polynucleotide. One of skill in the art can select the appropriate fluorescent proteins and inhibitory polynucleotide(s) to achieve the desired effect.

The target gene product and the fluorescent protein may be expressed from the same expression vector. In one embodiment the fluorescent protein gene and the target gene are cloned such that they are expressed as a fusion protein using any available means to fuse the coding regions for the two proteins, such as fusing the fluorescent protein to the amino- or carboxy-terminal of the target gene protein. In another embodiment the fluorescent protein gene and the target gene are expressed from a single message separated by an IRES (internal ribosome entry site) element. Expression vectors for expression of the fluorescent protein gene and target gene as a fusion protein, and expression vectors containing an IRES elements and a fluorescent protein gene are commercially available from vendors including Clontech, Invitrogen, and Stratagene.

Protein variants means proteins that may contain one or more substitutions, additions, deletions and/or insertions such that the therapeutic, antigenic and/or immunogenic properties of the proteins encoded by the variants are not substantially diminished, relative to the corresponding protein. Such modifications may be readily introduced using standard mutagenesis techniques, such as oligonucleotide directed site-specific mutagenesis as taught, for example, by Adelman et al. (DNA, 2:183, 1983). Preferably, the antigenicity or immunogenicity of a protein variant is not substantially diminished. Variants also include what are sometimes referred to as “fragments” that retain the biological activity of the protein. The term also includes proteins that may contain one or more amino acid substitutions, additions, deletions and/or insertions, such that the therapeutic, antigenic and/or immunogenic properties of the peptide variants are not substantially diminished, relative to the corresponding protein.

As used herein, “protein” includes “polypeptide” and means any chain of amino acids, regardless of length or post-translational modification (e.g., glycosylation or phosphorylation).

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. The contents of all references, pending patent applications and published patents, cited throughout this application are hereby expressly incorporated by reference. Although specific terms are employed, they are used as in the art unless otherwise indicated.

What is claimed is:

1. A method for testing the efficiency of delivering a first inhibitory polynucleotide to a host animal, comprising (a) introducing target cells capable of forming a target cell mass and expressing a first fluorescent protein into the host animal at a target site; (b) delivering the first inhibitory polynucleotide against the first fluorescent protein to the host animal; and (c) measuring over a time period in vivo the first fluorescent protein fluorescence to determine the efficiency of delivering the first inhibitory polynucleotide to the target cells, wherein the efficiency is inversely proportional to a reduction in fluorescence.

2. The method of claim 1, further comprising (d) delivering an expression construct comprising a second fluorescent protein to the host animal; (e) delivering a second inhibitory polynucleotide against the second fluorescent protein to the host animal; and (f) measuring over a time period in vivo the second fluorescent protein fluorescence to determine the efficiency of delivering the second inhibitory polynucleotide to the cells surrounding the target site, wherein the efficiency is inversely proportional to a reduction in second fluorescent protein fluorescence.

3. The method of claim 1, wherein the target cells comprise a target gene, and the method further comprises (d) delivering a second inhibitory polynucleotide against the target gene to the host animal; and (e) determining that the second inhibitory polynucleotide is successfully delivered to the target cells if the first fluorescent protein fluorescence measured in step (c) is reduced.

4. The method of claim 1, wherein the host animal is selected from the group comprising a nude mouse, a SCID mouse, a thymectomized mouse, or an irradiated mouse.

5. The method of claim 1, wherein the target cells are injected subcutaneously, intraperitoneally or at an orthotopic site.

6. The method of claim 1, wherein the inhibitory polynucleotide is delivered to the target site or at a remote site.

7. The method of claim 2, wherein the first and second inhibitory polynucleotides are delivered at the target site or at a remote site either simultaneously with or within 30 minutes of each other.

8. The method of claim 3, wherein the first and second inhibitory polynucleotides are delivered at the target site or at a remote site either simultaneously with or within 30 minutes of each other.

9. The method of claim 3, further comprising (f) determining the effect of the second inhibitory polypeptide against target gene on the target cells relative to a control.

10. The method of claim 1, wherein the inhibitory polynucleotide is an siRNA, shRNA, microRNA, antisense RNA, or a morpholino.

11. The method of claim 1, wherein the first inhibitory polynucleotide is delivered as a naked inhibitory polynucleotide, or packaged in a liposome, nanoparticle, virus, bacteria, or in a donor cell.

12. The method of claim 11, wherein the donor cell expresses one or more connexin proteins.

13. The method of claim 11, wherein the donor cell is an immune privileged cell.

14. The method of claim 11, wherein the donor cell is a mesenchymal stem cell.

15. The method of claim 1, wherein the inhibitory polynucleotide is complexed with cationic lipids, cholesterol, peptides, polyethyleneimine, or condensing polymers.

16. The method of claim 1, wherein the inhibitory polynucleotide has one or more chemical modifications selected from the group comprising changes to the inhibitory polynucleotide backbone, replacement of one or more nucleotides with nucleotide analogues, and addition of conjugates to the polynucleotide.

17. The method of claim 16, wherein the chemical modification is selected from the group comprising inclusion of a 2′O-methyl RNA, phosphorothioate bonds, linked nucleic acids, and/or addition of a moiety such as cholesterol, peptide, polyethylene glycol, or fatty acid.

18. The method of claim 1, wherein the first fluorescent protein is selected from the group comprising Green fluorescent protein; a far-red emitting protein; a red protein; an orange protein; a yellow protein; a yellow-green protein; a green protein; a cyan protein; or a UV excitable green protein; and biologically active fragments or variants thereof.

19. The method of claim 3, wherein the target gene is expressed endogenously in the target cells.

20. The method of claim 3, wherein the target cells have been engineered to express the target gene.

21. The method of claim 20, wherein the target gene is expressed from a plasmid, or from vectors including retrovirus, lentivirus, adenovirus, and adeno-associated virus based systems.

22. The method of claim 1, wherein the target cells are tumor-forming cells.

23. The method of claim 1, wherein the target cells form a target cell mass comprising one or more tumors.

24. The method of claim 3, wherein the target cells form a target cell mass and the effect of the second inhibitory polypeptide on the target cell mass is a reduction in size, a change in growth, or a change in metastasis.

25. The method of claim 1, wherein the target cells expresses a target gene and a second fluorescent protein.

26. Claim 1, wherein the target cells express the first fluorescent protein and a target gene on the same messenger RNA, and the method further comprises (d) determining that a reduction in first fluorescent protein fluorescence indicates a reduction in expression of the target gene.

27. The method of claim 27, wherein the first fluorescent protein and the target gene are expressed as a fusion protein in the target cells.

28. The method of claim 27, wherein the first fluorescent protein and the target gene are separated by an IRES element on the messenger RNA

29. The method of claim 27, wherein the target gene and the first fluorescent protein are expressed from a plasmid, or from a vector selected from the group comprising a retrovirus, lentivirus, adenovirus, and adeno-associated virus.

30. The method of claim 1, wherein the host animal is immune-compromised.

31. The method of claim 1, wherein the host animal is Syngenic.

32. The method of claim 1, further comprising repeating (a), (b) and (c) in additional host animals of the same type using different methods for delivering the first inhibitory polypeptide against the first fluorescent protein in each animal, and selecting the delivery method with the highest efficiency.

33. The method of claim 1, wherein the host animal is an immune-compromised transgenic animal that expresses a second fluorescent protein, and the method further comprises (d) delivering a second inhibitory polynucleotide against the second fluorescent protein to the host; and (e) measuring over a time period in vivo the second fluorescent protein fluorescence to determine the efficiency of delivering the second inhibitory polynucleotide to the host animal cells, wherein the efficiency is inversely proportional to a reduction in second fluorescent protein fluorescence.

34. The method of claim 1 wherein the target cells form a target cell mass before the first inhibitory polynucleotide is delivered.
